(19) United States
(12) Patent Application Publication
(10) Pub. No.: US 2005/0070784 A1
(43) Pub. Date: Mar. 31, 2005

(54) Magnetic resonance imaging apparatus and magnetic resonance imaging method

(76) Inventors: Kazumi Komura, Chiba (JP); Tetsuhiko Takahashi, Saitama (JP); Hisako Nagao, Chiba (JP)

Correspondence Address: COOPER & DUNHAM, LLP, 1185 AVENUE OF THE AMERICAS, NEW YORK, NY, 10036, US

(21) Appl. No.: 10/495,726

(22) PCT Filed: Nov. 15, 2002
(86) PCT No.: PCT/JP02/11931

(30) Foreign Application Priority Data
Nov. 15, 2001 (JP) 2001-350174

(51) Int. Cl. A61B 5/05

(52) U.S. Cl. 600/410; 600/415

(57) ABSTRACT

A magnetic resonance imaging apparatus includes control means 107 and 108 for continuously performing magnetic resonance imaging of a cross section including a portion subjected to measurement of an examinee (101) at a predetermined time interval, operation means 108 for calculating diagnosis information related to the portion subjected to measurement by using a plurality of sets of nuclear magnetic resonance signals related to cross sections imaged at different time points by measuring nuclear magnetic resonance signals generated from the examinee 101, and a position detection device 118 having a detection camera 118b for detecting in non-contact manner the position (three-dimensional position and rotation angle around an orthogonal coordinate axis) of a pointer 118a provided outside the examines body and moving while interlocked with the biological movement of the examinee 101. The control means 108 sets the position of the cross section according to the position of the pointer 118a detected by the position detection device 118, thereby eliminating an affect of the biological movement and improving the accuracy and the reliability of the diagnosis information including differential processing of a portion subjected to measurement.

### TECHNICAL FIELD

The present invention relates to a magnetic resonance imaging (hereinafter, referred to as MRI) apparatus and a magnetic resonance imaging method. Specifically, the present invention relates to setting a slice of the magnetic resonance imaging that is suitable for performing the continuously imaging of a portion subjected to measurement of an examinee which is moved by biological movement such as breath movement.

### BACKGROUND ART

An MRI apparatus excites particular atomic nuclei (for example, protons) constituting an examinee by applying radio-frequency magnetic field pulse (hereinafter, referred to as an RF pulse) together with a gradient magnetic field for setting the slice to the examinee placed In a static magnetic field. The MRI apparatus reconstructs a tomogram inside the examinee based on a nuclear magnetic resonance (NMR) signals generated by the excitation to provide the tomogram with the diagnosis The MRI apparatus is used to set a slice including an object portion of diagnosis, to continuously obtain tomograms of the slice in a time series, for obtaining various types of information necessary for diagnosis based on the tomograms obtained at different time points.

For example, in recent years, attention is paid to an interventional MRI apparatus (hereinafter, referred to as an IVMR apparatus) in which the MRI apparatus is used for monitoring during an operation. Treatments using the IVMR are, for example, a laser treatment, coagulation by microwave, injection of medicine such as ethanol, RF irradiating resection, and a low temperature treatment. In these treatments, the MRI apparatus plays role as a real time imaging guide for causing a inserting needle and a thin tube to reach an affected part, a monitor for visualizing a change of a tissue being treated, a temperature monitor of a portion subjected to a heating or cooling treatment, and the like. As an example of a typical application of the IVMR, it is mentioned to obtain an image of temperature distribution of a treated portion of the like of an examinee during a laser irradiation treatment and microwave coagulation.

The method of obtaining the image of temperature distribution described above includes a method of obtaining the signal intensity, a method of obtaining from a diffusion coefficient, a method of obtaining from the phase shift of protons (PPS. i.e. proton phase shift method), and the like. That is, the temperature is measured in use of the property that signal intensity from a tissue is changed in response to the temperature or the property that a diffusion coefficient of the Brownian movement of water and the like constituting a tissue is affected by temperature, wherein the PPS method demonstrates the best measuring accuracy.

In the FPS method, the temperature distribution is determined from the phase information of echo signals obtained by, for example, inversion of gradient magnetic field. Specifically, a phase distribution is determined from the real part Sr and the imaginary part Si of a complex image obtained by subjecting the echo signals to Fourier transformation using the following equation (1).
φ(x, y, z)=tan^{31 1}(Si(x, y, z)/Sr(x, y, z)) (1)

Then, the temperature T in the following equation (2) is determined from thus obtained phase distribution, an interval (echo time) TE between a timing of the maximum echo signal and a 90° pulse, a resonance frequency f, and a water temperature coefficient.
T[° C.]=φ[°]/{TE[s]*f[Hz]*0.01[ppm/° C.]*10⁻⁶*360[°]} (2)

A distribution of temperature change of the examinee at a certain time can be obtained by determining the difference between the temperature distributions calculated from the signals obtained at different time points t1 to tn (n is the number of imaging time points) using the above method.

As described above, when the temperature is monitored by MRI, the same temperature change region (a region subjected to measurement) must be constantly in order to obtain continuous time series data, to determine the difference between the spatial phase distributions obtained at different time points, and to determine a temperature change. However, when an imaging cross section is fixed in a space, the region subjected to measurement is often out of the imaging cross section movement of the examinee, in particular, in the abdomen, by an affect of breath movement. Therefore, it is difficult to stably image the region subjected to measurement. For example, the thickness of the imaging cross section(slice) is in the order of several millimeters to ten millimeters, whereas the breath movement is in the range of 10 mm or more in an interval of about three seconds. Thus, it may occur that a tomogram measured at a time phase includes the region subjected to measurement, and a tomogram measured at another time phase does not include the region. Accordingly, in case of a heat treatment, the measured time series date includes both data having information of temperature increase in a heated portion and data without the information of temperature increase. In the latter case, the information of temperature increase caused by heating cannot be obtained. To cope with the above problem, when it is intended to measure and display a temperature change in real time using a difference between the time series data. The temperature sometimes increases and sometimes does not increase, or in some cases a heating region suddenly expands or disappears, whereby the temperature cannot be stably monitored. Therefore, the result lacks reliability.

The above problem in measurement caused by movement of an examinee is a common problem when diagnosis is executed by measuring continuously and in time series the NMR signals from an imaging cross section including a region subjected to measurement and comparing the measured data of a plurality of imaging cross section obtained at different times, in addition to the measurement of the temperature distribution described above. For example, MR angiography known as a blood vessel imaging method includes an image processing to improve the contrast of a particular portion such as a blood vessel by determining the difference between two blood vessel images obtained at offset times. In this case, when the positions of blood vessel in the two images are displaced by movement, there is a problem such that a blood vessel Is blurred. In a conventional teaching, in such a case, the positional displacement of the two images is corrected by determining the amount of positional displacement based on the feature of the positional displacement that appears in a differential image (refer to JP2001-252262A). However, since the correction is executed after obtaining the images, it cannot be applied to a case where real time processing is required. Further, as another example of the MR angiography, blood flow information is measured and drawn by taking an image of a plurality of blood vessel cross sections while moving a slicing plane in parallel along the extending direction of a blood vessel (refer to JP2002-253527A). In this-case, when the positions of blood vessel in images are displaced by body movement, a measurement error arises. That is, when a portion subjected to measurement is located outside a field of view, the portion cannot be used for comparison, or when the positions of the portions subjected to measurement in the images are relatively displaced, differential image includes an error.

Accordingly, a first object of the present Invention is to enable positioning of an imaging cross section in conformity with movement of a portion subjected to measurement caused by body movement when the portion subjected to measurement is continuously taken as image.

Further, a second object of the present invention is to improve accuracy and reliability of temperature monitoring by avoiding an affect of body movement when the distribution of temperature changes of a particular portion such as a treatment portion is measured.

### DISCLOSURE OF THE INVENTION

To achieve the first object, there is provided a magnetic resonance imaging method of the present invention comprising the steps of : taking time series images of a measuring cross section including a portion subjected to measurement of an examines using a magnetic resonance imaging, and obtaining diagnosis information by comparing magnetic resonance signals related to the plurality of the measuring cross sections obtained above in an operation process, wherein a body movement of the examinee is detected and a position of the movement of the examinee is detected and a position of the measuring cross section is set so as to include the portion subjected to measurement in conformity with the detected body movement.

A magnetic resonance imaging apparatus of the present invention for embodying the imaging method includes a means for generating a uniform static magnetic field in a space where an examinee is placed, a means for generating a gradient magnetic field for determining an image-taking cross section of the examines, a means for applying a radio-frequency magnetic field to the space, a means for detecting nuclear magnetic resonance signals generated from the examinee, a control means for continuously executing magnetic resonance imaging of the image-taking cross section including the portion subjected to measurement of the examinee at time intervals, a means for operating the diagnosis information of the portion subjected to using a plural sets of nuclear magnetic resonance signals of the image-taking cross section detected by the detection means and executed at different time points, and a means for displaying the diagnosis information, further including a body movement detection means for detecting a body movement of the examinee, and the control means sets the position of the image-taking cross section based on the information from the body movement detection means.

In this case, the control means can determine the position of the portion subjected to measurement based on the information from the body movement detection means and set the position of the above determined portion subjected to measurement.

First, the body surface or a body surface portion (hereinafter, simply referred to as the body surface and the like) of the examinee is moved in correlation to the body movement of the examinee moved by his breath and the like. Further, a certain correlation exists between a movement of the body surface and the like and a movement of the portion subjected to measurement in the examinee. Accordingly, it is possible to detect the position of a pointer moving in association with, for example, the body surface and the like in real time and to detect the movement of the portion subjected to measurement by calculation executed based on the above correlation. The movement of the portion subjected to measurement is expressed by a change of a three-dimensional position or by a change of a three-dimensional position and a rotation angle about orthogonal coordinate axes (hereinafter, referred to as a six-dimensional position). Then, the position of the image-taking cross section is set so that the portion subjected to measurement is located at the same position by moving the image-taking cross section in parallel or along the image-taking cross section in conformity with the three-dimensional position of the portion subjected to measurement having been detected. As known well, the setting is conducted by adjusting the gradient magnetic fields in the direction of three orthogonal axes. Further, when the six-dimensional position is detected, the gradient angle of the image-taking cross section is set with respect to, for example, a body axis, in addition to the setting of the three-dimensional position of the image-taking cross section.

As described by setting the position of the image-taking cross-section in the magnetic resonance imaging continuously executed with time intervals in real time in conformity with the movement of the portion subjected to measurement, it is possible to eliminate an error in the operation process for obtaining necessary diagnosis information by comparing the nuclear magnetic resonance signals related to the plurality of image-taking cross sections obtained at different time points.

To achieve the second object, the operation means is characterized by having a function for determining the temperature or the temperature distribution of the portion subjected to measurement based on the nuclear magnetic resonance signals, determining the temperature or the temperature distribution difference of the same portion subjected to measurement according to the slices at different times, and determining the temperature change or the temperature change distribution of the portion subjected to measurement.

With the above function, it is possible to improve the accuracy and the reliability in temperature measurement. Further, the temperature change of the portion subjected to measurement can be monitored by displaying the thus-determined temperature change distribution as a color image.

Further, as another example of the operation means for determining the necessary diagnosis information, there are an operation process for reconstructing an MR image of a blood vessel image and the like of a portion subjected to measurement based on nuclear magnetic resonance signals and creating a differential image of the MR images of the blood vessel image and the like of the same portion subjected to measurement at different time points, and the like. In this case, it is possible to improve image quality by refining the blur of the blood vessel image and the like.

Next, examples of the biological movement detection means for detecting the biological movement will be specifically explained.

(1) A position detection means is arranged by disposing a pointer on the body surface of the examinee or in relation to the body surface and disposing a plurality of detectors at positions apart from the pointer. Then, signals are transmitted and received between the plurality of detectors and the pointer through a space, and the position of the pointer is detected based on the positional relation between the plurality of detectors and the pointer. A known position detection device can be used as the position detection means, and when the position detection means is classified in principle, there can be applied a position detection means employing a system that detects the position of the pointer by transmitting and receiving light signals, ultrasonic wave signals, electromagnetic wave signals, and the like between the detectors and the pointer.

(2) As a position detection means using light, there is a position detection means that uses, as the pointer, a reflector for reflecting light and has a light emitter and two cameras disposed apart from the pointer and detects the three-dimensional position of the pointer based on the two images formed by receiving the light of the light emitter, which is reflected by the reflector, by the two cameras.

(3) As another example of the position detection means using light, there is a position detection means that uses, as the pointer, three reflectors for reflecting light disposed at the apexes of a triangle and has a light emitter and two cameras disposed apart from the pointer and detects the three-dimensional position and the rotation angle about an orthogonal coordinate axis of the pointer based on the two images formed by receiving the light of the light emitter, which is reflected by the reflectors, by the two cameras. As an example of the position detection system, there Is known POLARIS (commodity name) of Northern Digital Instrument.

Further, the pointer is fixed in contact with a body surface near to a portion subjected to measurement or fixed to a needle device inserted into an examinee at a position outside of the examinee (for example, the rear end of the needle device). When the three reflectors are disposed discretely, it is preferable to dispose them at the apexes of a triangle. The needle device is composed of a laser fiber passed through a guide inserted into a treatment portion for warming it, an electrode inserted into a treatment portion for irradiating micro waves thereto, and the like. When any of these needle devices is used, the temperature of the needle device is measured at the extreme end thereof. In this case, although the needle device may be turned about its axis, it is not necessary to turn a slice in accordance with the turn of the needle device. Thus, it is preferable to execute such a correction as to extract the rotation angle component about the axis of the needle device from the rotation angle about an orthogonal coordinate axis of the pointer detected by the position detection means and to subtract the rotation angle component about the axis of the needle device from the rotation angle about an orthogonal coordinate axis of the pointer.

Further, it is preferable to previously determine the correlation data between the movement of the portion subjected to measurement caused by biological movement and the movement of the pointer and to determine the three-dimensional position and the rotation angles about the orthogonal coordinate axes of the portion subjected to measurement from the three-dimensional position and the rotation angles about the orthogonal coordinate axes detected by the position detection means based on the correlation data.

### BRIEF DESCRIPTION OF THE DRAWINGS

FIG. 1 is a view showing the overall arrangement of an MRI apparatus to which the present invention is applied.

FIG. 2 is a view showing an important portion of a position detection device.

FIG. 3 is a flowchart showing an embodiment related to a temperature measuring procedure by the MRI apparatus according to the present invention.

FIG. 4 is a view showing an example of a pulse sequence employed in temperature measurement.

FIGS. 5(a) to 5(d) are views explaining temperature measurement according to the present invention.

FIG. 6 is a graph schematically showing a change of a temperature change region due to body movement.

FIG. 7 is a view showing another embodiment of the temperature measurement according to the present invention.

### BEST MODE FOR CARRYING OUT THE INVENTION

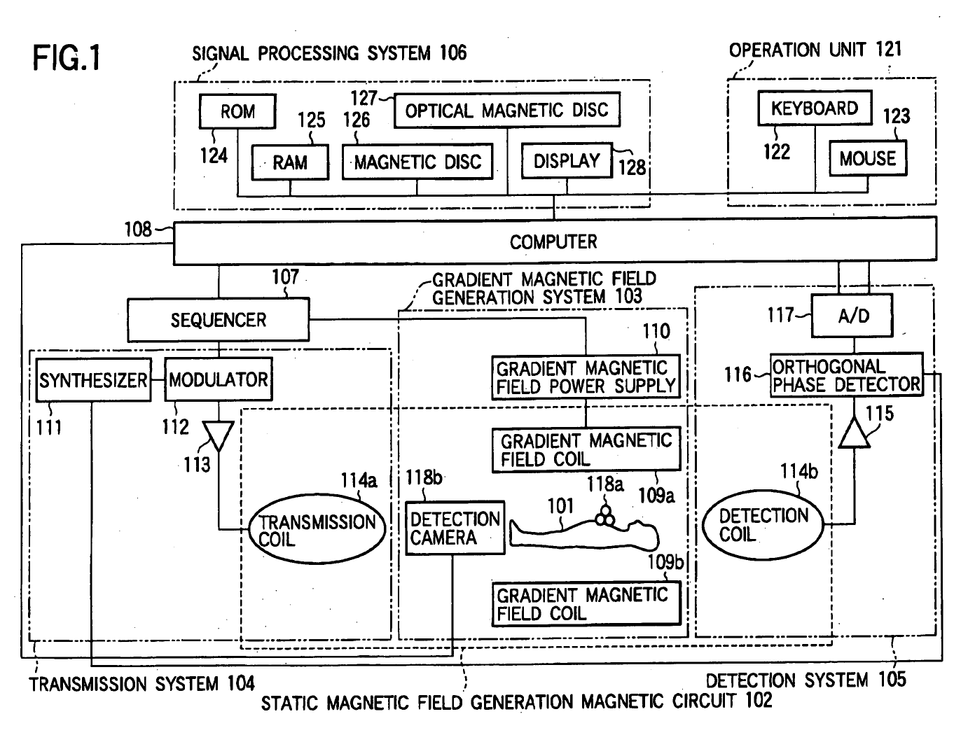

An embodiment of an MRI apparatus of the present invention will be described below with reference to the drawings. FIG. 1 is a view showing the overall arrangement of the MRI apparatus to which the present invention is applied. The MRI apparatus includes a static magnetic field generation magnetic circuit 102 composed of an electromagnet or a permanent magnet for generating a uniform static magnetic field H0 in an examinee 101, a gradient magnetic field generation system 103 for generating gradient magnetic fields Gx, Gy, Gz the intensities of which linearly change in three axis directions which are orthogonal to each other, a transmission system 104 for applying a radio-frequency magnetic filed (RF pulses) to the examinee 101, a detection system 105 for detecting NMR signals generated from the examinee 101, a gradient magnetic field generation system 103, a sequencer 107 for transmitting a command to the transmission system 104 and the detection system 105 and generating gradient magnetic fields and the RF pulses at a predetermined timing, a computer 108 for executing various processings such as the control of the sequencer 107, image processing, and temperature calculation, a signal processing system 106 for displaying and storing an image, an operation unit 121 including a keyboard 112 and a mouse 123 for executing operations for setting various parameters such as imaging conditions to the computer 108, and a position detection device 118 including pointers 118a and a detection camera 118b for detecting the position of a particular portion of the examinee 101 lying on a bed.

The gradient magnetic field generation system 303 includes gradient magnetic field coils 109a and 109b in the three axis directions and a power supply 110 thereof, determines a slice of the examinee 101 depending on a manner of applying gradient magnetic fields, and provides the NMR signals generated by the examinee 101 with position information. In the present invention, the gradient magnetic fields for determining the slice are controlled based on the position information from the position detection device 118 through the computer 108.

The transmission system 104 includes a synthesizer 111, a modulator 112, a power amplifier 113, and a transmission coil 114a, modifies the radio-frequency generated by the synthesizer 111 through the modulator 112 at the timing commanded by the sequencer 107, amplifies the thus modified radio-frequency through the power amplifier 113, and supplies it to a transmission coil 114a. In the above operation, a radio-frequency magnetic field is generated in the examinee 101, and nuclear spin is exited.

The detection system 105 includes composed of a detection coil 114b, an amplifier 115, an orthogonal phase detector 116, and an A/D converter 117, receives the NMR signals generated from the examinee 101 by the detection coil 114b, amplifies the NMR signals by the amplifier 115, detects the signals by the orthogonal phase detector 116 with reference to the reference radio-frequency signals from the synthesizer 111, and inputs NMR signals to the computer 108 as a two sets of digital signals.

Although the transmission coil 114a and the detection coil 114b are separately provided in the figure, it is also possible to use a single coil having both a transmission function and a reception function.

After the computer 108 subjects the signals input from the detection system 105 to predetermined signal processing, it calculates a nuclear spin density distribution, a relaxation time distribution, a spectrum distribution, a temperature distribution, and the like and creates an image. Further, in the present invention, the computer,108 captures the signals as to the position information of a portion subjected to measurement of the examinee from the position detection device 118, determines the position of a slice based on the position information, and outputs a command for generating gradient magnetic fields corresponding to the slice to the sequencer 107.

The image formed by the computer 108 is displayed on a display 128 of the signal processing system 106 and stored in a magnetic disc 126, an optical magnetic disc 127, and the like when necessary. Meanwhile, the signal processing system 106 has a ROM 124 and a RAM 125 storing data in course of calculation, various parameters necessary for calculation, and the like.

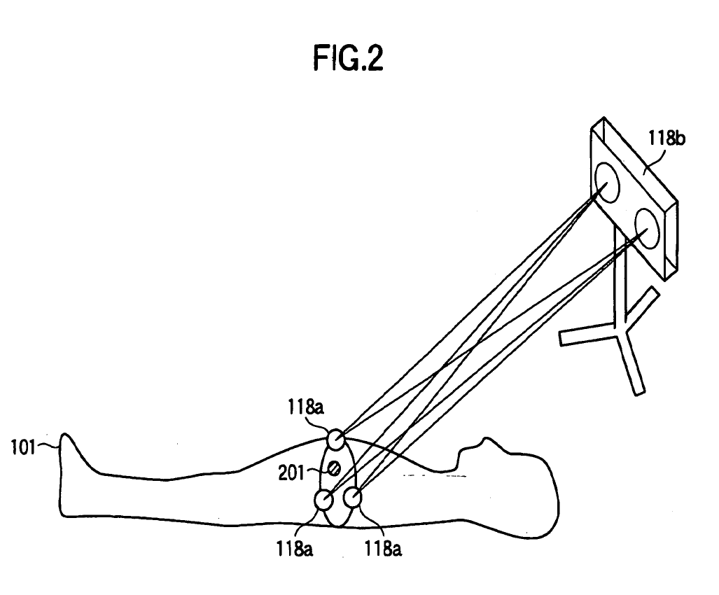

The position detection device 118 detects a particular region of the examinee 101, specifically, a position (coordinate) in the measurement space of an object region where a temperature change is measured. Signals as to the information of a position detected by the position detection device 118 are transmitted to the computer 108 through a line. The computer 108 determines the position of the slice of the examinee 101 based on the signals as to the information of the detected position. As shown in FIG. 2, for example, the position detection device 118 includes pointers 118a (three sets in the illustrated example) fixed on the body surface of the examinee 101 in the vicinity of an object measuring region 201 and a detection camera 118b having two cameras for detecting the positions of the pointers 118a in order to indicate the specific region of the examinee 101. A known pointer developed to obtain an MR image as to a desired position can be used as the pointer 118a. Specifically, an active- or passive-type pointer having at least three infrared ray emitting diodes or reflection bulbs disposed at the apexes of a triangle can be used. The passive-type diodes are preferable in operability because they do not need a power supply line. The detection camera 118b is composed of at least two cameras attached at positions having a parallax with respect to the pointers, and when the passive-type pointers using the reflection bulbs are used, the cameras are provided with light emitting diodes acting as light emitters for illuminating the reflection bulbs. The detection camera 118b is disposed at a position, i.e. 1 m to 1.5 m apart from the center of the static magnetic field generating region of the MRI apparatus.

The pointers 118a can be disposed at a predetermined portion such as a treatment portion and the rear end (portion remaining outside of the examinee's body) of an instrument (for example, an Inserting needle and an inserting guide) that is inserted into the examinee 101, other than on the body surface of the examinee 101. Then, the two cameras detect the positions of the respective light emitting diodes or the reflection bulbs of the pointers 118a in real time and send the six-dimensional position information of the center points of the pointers 118a (that is, rotational information as to x-, y-, and z-axes) to the computer 108 in real time. A position detection device POLARIS of Northern Digital Instrument, for example, can be used as the position detection device 118 arranged as described above, and a detection speed of 20 to 60 MHz and a position accuracy of 0.35 mm can be realized by the position detection device. Meanwhile, when only the three-dimensional position of the pointer 118a is detected, the pointer 118a can be formed of one light emitting diode or one reflection bulb.

Meanwhile, although not illustrated, a reference pointer is disposed at a fixed position apart a predetermined distance from the center of a magnetic field to convert the positions of the pointers 118a into a coordinate from the center of the magnetic field in the measurement space of the MRI apparatus. That is, as an initial operation, the position of the reference pointer is detected by the detection camera 118b, the position of the reference pointer, for example, is determined as the point of origin of a measurement space coordinate, and the coordinates of the positions of the pointers 118a in the measurement space coordinate are detected.

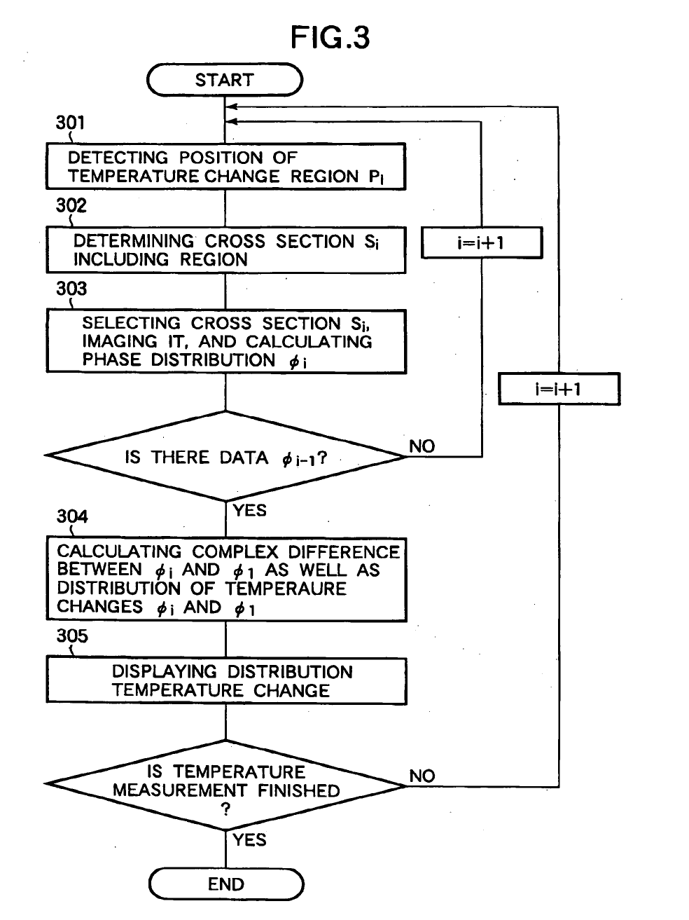

Next, a temperature measuring method executed using the MRI apparatus will be explained with reference to FIGS. 3 to 5. The temperature measurement using the MRI apparatus is applied when treatments such as a laser treatment, coagulation by microwave, injection of medicine such as ethanol, RF irradiating resection, and a low temperature treatment are executed, and when a simple operation is executed by IV-MR in order to monitor the local temperature of an object portion during the treatments or the operation.

First, as shown in FIG. 2, the pointers 118a of the position detection device 118 are disposed in the vicinity of the object temperature change region 201 on the body surface of the examinee 101 placed in the measurement space and the detection camera 118b starts to measure the positions of the pointers 118a in real time. Next, the detection camera 118b starts to photograph a cross sectional surface S1 including the temperature change region 201. The cross sectional surface S1 that is imaged first is determined by imaging and displaying an image along, for example, the body axis direction of the examinee and selecting a cross sectional surface including an object portion from the image, similar to a case that an ordinary image is taken. A gradient magnetic field corresponding to the cross sectional surface S1 selected as described above is determined, and the thus determined gradient magnetic field is set as a parameter of a slice.

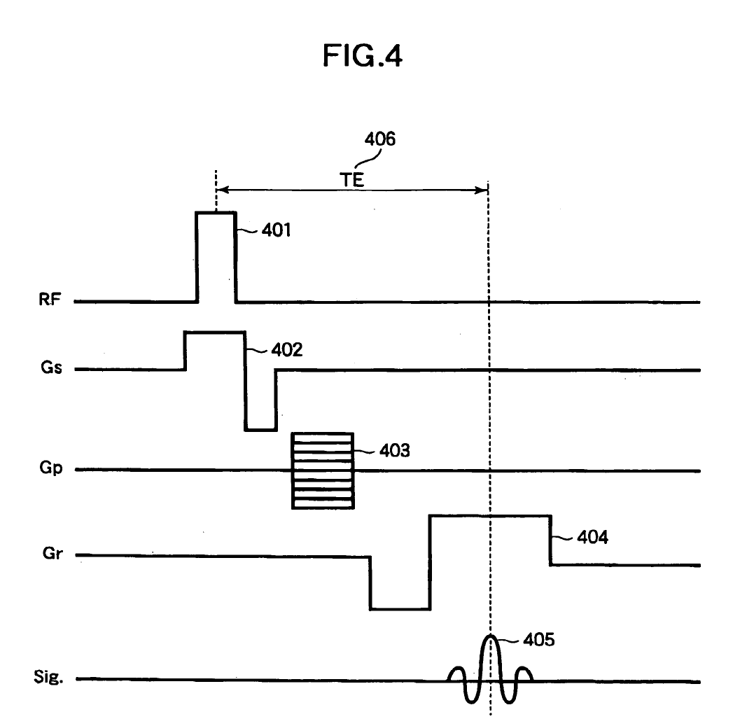

Image-taking is executed by the pulse sequence of a gradient echo method (GRE) as shown in, for example, FIG. 4. That is, a gradient magnetic field Gs 402 for selecting the slice is applied together with radio-frequency magnetic field pulses (RF pulses) 401, then a phase encode gradient magnetic field Gp 403 is applied, and a gradient echo Sig. 405 is measured while applying a read-out gradient magnetic field Gr 404 having an inverting polarities. The sequence is executed repeatedly while changing the intensity of the phase encode gradient magnetic field Gp 403 and measuring a set of the signals including temperature information of the slice. A phase distribution φ1(x, y, z) is determined from the real part and the imaginary part of complex image data, which is obtained by subjecting the echo signals to Fourier transform, using the above equation (1).

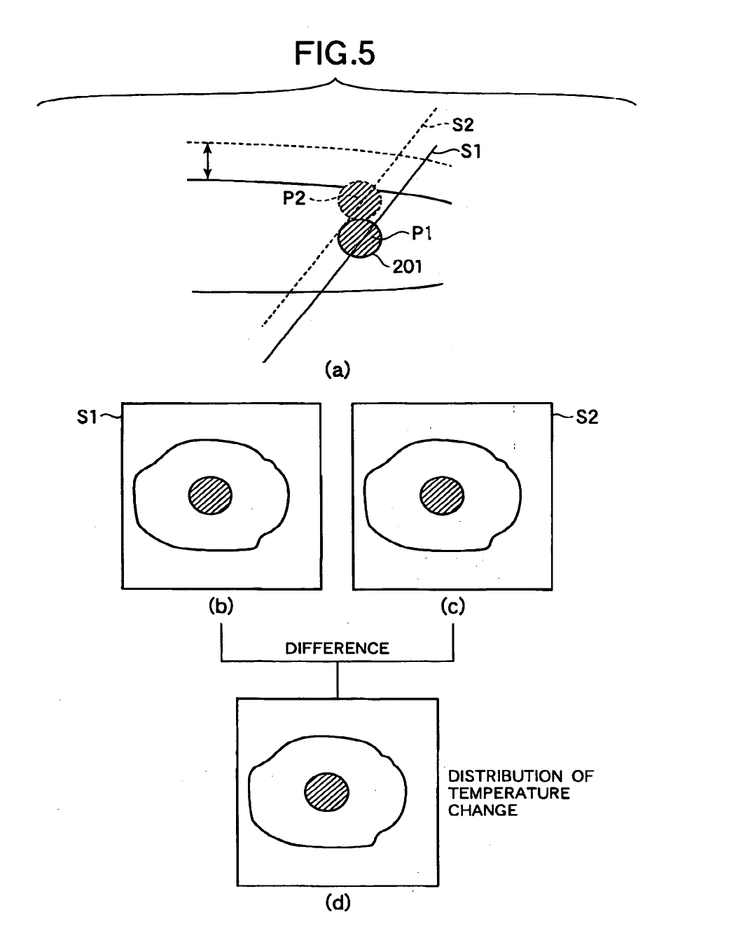

As shown in FIG. 5(b), the image of the thus obtained phase distribution reflects the temperature information of the slice S1 including the temperature change region 201. A time at which the image of the phase distribution Is set to t1, the same measurement is executed at a time t2 after Δt passes from the time t1. However, in this case, the position of the temperature change region 201 changes from a position P1 at the time t1 to P2 by breath movement as shown in FIG. 5(a). The computer 108 captures the six-dimensional position information of the pointers 118a from the position detection device 118 (step 301 of FIG. 3), calculates the slice S2 Including P2 by calculating the six-dimensional position of P2 and determines a gradient magnetic field Gs 402 for selecting the slice S2 (step 302). Then, a command is sent to the sequencer 107 so that the newly determined gradient magnetic field is used as the gradient magnetic field for selecting the slice when the pulse sequencer of FIG. 4 is executed. Then, at the time t2, the newly selected the slice S2 is measured (step 303).

Although the phase distributions φ1 and φ2 obtained at the times t1 and t2 described above select the different slices in the measurement space, they act as the phase distribution of approximately the same slice including the same temperature change region in the examinee being moved (FIG. 5(c)). The complex difference between these two phase distributions φ1 and φ2 is calculated, and a temperature change distribution ΔT is calculated based on the temperature difference (T1−T2) between the times t1 and t2 by an equation (3) (step 304).
ΔT−T1−T2=(φ1−φ2)/TE*f*0.01*10⁻⁶*360) (3)

The image of the thus obtained temperature change distribution (FIG. 5(d) is displayed on the display (step 305). Thereafter, the slice corresponding to the positions of the pointers is imaged at every predetermined time interval, and temperature change distributions are determined from a phase distribution φ1 calculated as to the slice and the phase distribution φ1 determined first and sequentially displayed on the display. An operator can execute a treatment such as warming while monitoring the image of the temperature change distribution displayed on the display.

Although the temperature change distribution is determined from the complex difference between the i-th phase distribution φ1 and the phase distribution φ1 determined first at step 304, a temperature change distribution Ti from the beginning of measurement may be determined by calculating a temperature change distribution ti by determining the complex difference between the i-th phase distribution φ1 and an (i+1)-th phase distribution φ (i+1) and cumulating the temperature change distribution ti (Ti−Σti). That is, a phase change of (φ1−φ1)>360° may occur, this method is effective when there is a large phase change.

Further, the example FIG. 5(a) shows a case that the temperature change region 201 simply moves up and down as shown by an arrow, that is, a case that the slices S1 and S2 move in parallel with each other. However, the embodiment is not limited thereto, and even if the position of the temperature change region 201 moves six-dimensionally, the gradient magnetic fields Gs 402, Gp 403, and Gr 404 can be set by determining the six-dimensional position (the three-dimensional position and the rotation angle about orthogonal coordinate axes) of the slice S2 based on the position information by which the six-dimensional positions of the pointers 118a are detected.

As described above, according to the embodiment, since a phase distribution image including the same temperature region can be obtained at all times even a slice is different in the space, the temperature change of the temperature change region whose temperature is measured can be securely monitored, thereby the accuracy of a warming treatment and the like can be improved. Furthers the temperature change of the portion subjected to measurement can be monitored by displaying the determined temperature change distribution as a color image.

Further, in the embodiment described above, the positions at which the pointers 118a are disposed and which are detected by the position detection device 118 are not the same as the position of the temperature change region 201. However, in the region, in which it can be regarded that the temperature change region is interlocked with the movement of the pointers 118a, the position of a slice can be calculated by regarding the movement of the pointers 118aas the movement of the temperature change region as it is.

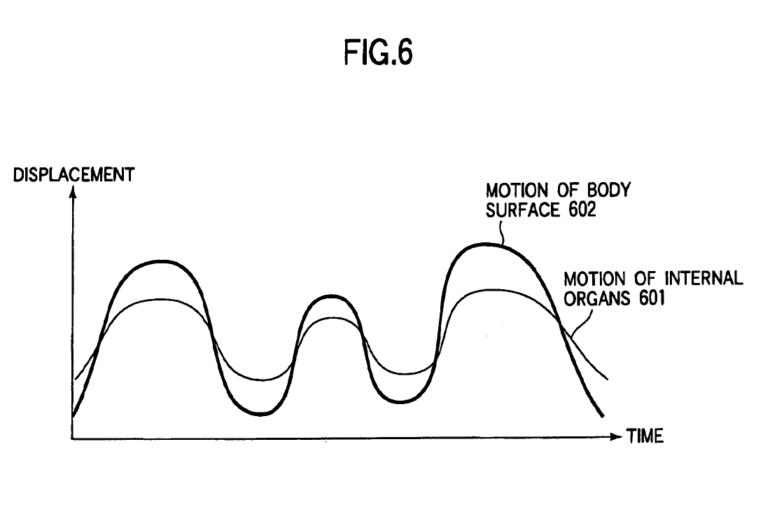

Meanwhile, when the variation 601 of the temperature change region is interlocked with a breath movement 602 but the amount of movement of the temperature change region is different from that of the breath movement 602 as shown in FIG. 6, a plurality of morphological images are previously obtained as to different time phases and the relation (displacement) between the amounts of movement is determined as shown in FIG. 6. It is possible to more accurately calculate the position of the temperature change region by the correlation data determined previously and the detected center positions of the pointers 118a. Namely, it is preferable to previously measure the correlation data between the movement of the portion subjected to measurement caused by biological movement and the movement of the pointers 118a and to determine the three-dimensional position and the rotation angle about an orthogonal coordinate axis of the portion subjected to measurement from the three-dimensional position and the rotation angles about the orthogonal coordinate axis detected by the position detection device 118 based on the correlation data. Further, when an internal organ acting as the temperature change region is exposed by an incision and the like, the movement of the temperature change region can be monitored at once by directly disposing the pointers 118a in the vicinity of the internal organ.

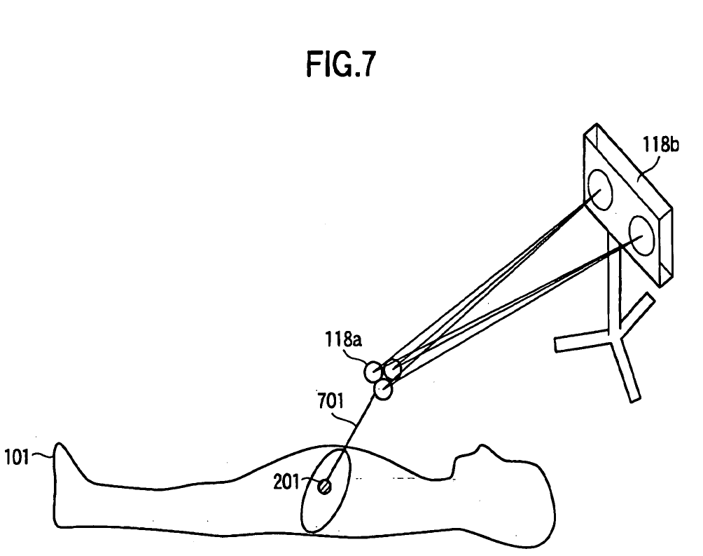

Further, when heating is executed by a laser fiber passed through an Inserted guide and when a microwave is irradiated from an inserted needle electrode, the pointers 118a can be disposed at the rear end of an inserting needle 701 as shown in FIG. 7. In this method, since the positional relation between the rear end and the front end of the inserting needle 701 is fixed, an extreme end position can be found by detecting a rear end position. Accordingly, the slice of the temperature change region can be selected by directly calculating the position of the temperature change region at the extreme end of the inserting needle 701 in the space.

Further, there are a heat device for heating a treatment portion by a laser fiber passed through an inserted guide, a device for irradiating a microwave to a treatment portion from an inserted needle electrode, and the like as needle devices which are used by being inserted into the examinee as in the inserting needle 701. When these needle devices such as the inserting needle 701 and the like are used, the extreme ends thereof act as object portions at which temperature is measured. In this case, the three-dimensional position and the rotation angle about an orthogonal coordinate axis of the extreme end of a needle device are determined based on the three-dimensional positions and the rotation angles about orthogonal coordinate axes of the pointers 118a detected by the position detection device 118, and the three-dimensional position and the rotation angle about an orthogonal coordinate axis of a slice are set such that the slice includes the axis of the needle device as well as the three-dimensional position and the rotation angle about an orthogonal coordinate axis of the slice agree with those of the needle device.

Further, although a needle device may be turned about its axis, it is not necessary to turn a slice in accordance with the turn of the needle device. Therefore, it is preferable to execute such a correction as to extract the rotation angle component about the axis of the needle device from the rotation angles about orthogonal coordinate axes of the pointers 118a detected by the position detection device 118 and to subtract the rotation angle component about the axis of the needle device from the rotation angles about orthogonal coordinate axes of the pointers 118a.

According to the embodiment of the present invention for executing the temperature measurement described above, even if a positional displacement is caused by the biological movement and the like in a region whose temperature is monitored, the temperature of the region can be accurately measured, thereby accuracy and reliability of the temperature measurement can be improved. The example of displaying the image of temperature distribution is explained in the above description, and, in this case, it is also possible to numerically display a temperature, a temperature difference, and the like, in addition to the image of temperature distribution.

Further, the present Invention is not limited to the imaging processing for measuring the temperature change and the distribution of temperature changes of a portion subjected to measurement and can be also applied to an imaging method including image processing for obtaining diagnosis information by comparing the MR images of the same portion subjected to measurement measured at different times. According to the imaging method, since the positional dislocation of the portion subjected to measurement can be reduced between the images to be compared, accuracy and reliability of the diagnosis information can be improved.

An embodiment in which the present invention is applied to MR angiography of a blood vessel will be explained. The MR angiography is a technology for drawing a blood flow image while improving the contrast of, for example, a particular portion by time series measuring the NMR signals related to a slice including a blood vessel and subjecting a plurality of blood vessel images of the slice at different times to differential processing, and various methods have been proposed (JP2001-252262A, JP2002-253527A). Even if any of the methods is employed, since two blood images of the same portion at different times are subjected to the differential processing, an error is included in the differential processing when the blood vessel and the like are moved by biological movement. As a result, although image quality is deteriorated by the blurring and the like of the blood vessel and the like, the error of the differential processing can be suppressed by reducing the positional displacement of the pair of blood vessel images subjected to the differential processing by setting the slice in conformity with the movement of the examinee or the blood vessel. Moreover, although the conventional method of detecting and correcting the positional displacement of the two blood vessel images is executed off-line after they are imaged, since the slice itself can be set in conformity with biological movement in the present invention. MR angiography imaging can be executed in real time.

A procedure of an embodiment for creating the three-dimensional differential image of an imaging region including an object imaging blood vessel by a method of using a contrast agent will be explained. First, before the contrast agent is injected, the region including the object imaging blood vessel is imaged by three-dimensional MRI. At the time, as shown in FIG. 2, the pointers 118a are fixed on the body surface of an examinee, the positional changes of the pointers 118a are detected, and the position and the orientation of a slice are set each time imaging is executed based on the correlation between the previously measured changes of the positions and orientations of the pointers 118a and the changes of the position and the orientation of the blood vessel. With the above operation, even if the blood vessel is moved by biological movement, the blood vessel is imaged at the same position and in the same orientation in an MR image. As described above, three-dimensional blood vessel images are collected before the contrast agent is injected. Next, after the contrast agent is injected, the region including the blood vessel is imaged by known three-dimensional MRI in exact timing with that a blood containing the contrast agent flows through an object imaging portion. At the time, the position and the orientation of a slice are set each time imaging is executed based on the correlation between the previously measured changes of the positions and orientations of the pointers 118a and the changes of the position and the orientation of the blood vessel as in the above. With this operation, the blood vessels in the MR images are imaged at the same position and in the same orientation before and after the contrast agent is injected. Accordingly, even if the blood vessel images of the same slice are subjected to the differential processing before and after the contrast agent is injected, the error of a differential image can be reduced because the position of the blood vessel is displaced little or is not displaced at all. As a result, a differential image, which is less blurred and has high quality, can be obtained.

As described above, according to the embodiment, there is an advantage that the MR angiography can be executed In real time because correction Is made against biological movement by changing a slice in conformity with blood movement during imaging.

It is needless to say that the present invention is by no means limited to the MR angiography employing the method of using a contrast agent and can be also applied to the imaging method as other MR angiography that is disclosed in, for example, JP2002-253527A in which blood flow information is measured and drawn by imaging a plurality of blood vessel cross sections while moving sliced surfaces in parallel with each other along the direction in which a blood vessel extends.

Further, the present invention is not limited to the above embodiment and may be variously modified. For example, although FIG. 4 exemplifies the sequence by the gradient echo method as the pulse sequence for measuring concentration, other sequences may be employed as long as they are GrE sequences which can obtain echo signals in which a phase component includes a temperature dependent component (resonance frequency×static magnetic field intensity), in addition to the sequence of FIG. 4. Specifically, known pulse sequences, for example, high speed GrE sequences such as SARGE, TRASARGE, and RFSARGE, sequences such as SSFP (Stredy State Free Precession), and the like, and GrP type EPI sequences can, be employed.

Further, although the above embodiment exemplifies the optical camera, and the optical devices such as the pointers and the like, which are imaged by the optical camera, a method of using an electromagnetic wave, and a method of using an ultrasonic wave can be appropriately used.

1. A magnetic resonance imaging apparatus comprising: means for generating a uniform static magnetic field in a space where an examinee is placed, means for generating a gradient magnetic field for determining a image-taking cross section of the examinee, means for applying a radio-frequency magnetic field to the space, means for detecting nuclear magnetic resonance signals generated from the examinee, control means for continuously executing magnetic resonance imaging of the image-taking cross section including the portion subjected to measurement of the examinee at time intervals, means for operating the diagnosis information of the portion subjected to using a plural sets of nuclear magnetic resonance signals of the image-taking cross section detected by the detection means and executed at different time points, and display means for displaying the diagnosis information, the magnetic resonance imaging apparatus, further comprising:
body movement detection means for detecting the body movement of the examinee, wherein the control means sets the position of the image-taking cross section based on the information from the body movement detection means.

2. A magnetic resonance imaging apparatus according to claim 1, wherein the control means determines the position of the portion subjected to measurement based on the information from the body movement detection means and sets the position of the cross section in conformity with the position subjected to measurement.

3. A magnetic resonance imaging apparatus according to claim 1 or 2, wherein the body movement detection means comprises a pointer disposed in association with the examinee and a detector for detecting the signals from the pointer, and the detector detects the body movement of the examinee based on the positional relation between the detector and the pointer.

4. A magnetic resonance imaging apparatus according to claim 1, wherein the body movement detection means includes position detection means for detecting the three-dimensional position of the pointer disposed in association with the examinee, and the control means sets the three-dimensional position of the cross section by determining the three-dimensional position of the portion subjected to measurement based on the three-dimensional position of the pointer detected by the position detection means.

5. A magnetic resonance imaging apparatus according to claim 3, wherein the body movement detection means detects the three-dimensional position and the rotation angle about an orthogonal coordinate axis of the pointer, and the control means sets the three-dimensional position and the rotation angle about an orthogonal coordinate axis of the cross section by determining the three-dimensional position and the rotation angle about an orthogonal coordinate axis of the portion subjected to measurement based on the three-dimensional position and the rotation angle about orthogonal coordinate axis of the pointer detected by the body movement detection means.

6. A magnetic resonance imaging apparatus according to claim 3, wherein the body movement detection means includes position detection means that comprises a pointer having a reflector for reflecting light and a light emitter and two cameras disposed apart from the pointer and detects the three-dimensional position of the pointer based on the two images formed by receiving the light from the light emitter, which is reflected by the reflector, by the two cameras; and
the control means sets the three-dimensional position of the cross section by determining the three-dimensional position of the portion subjected to measurement based on the three-dimensional position of the pointer detected by the position detection means.

7. A magnetic resonance imaging apparatus according to claim 3, wherein the body movement detection means includes position detection means including a pointer having three reflectors for reflecting light disposed at the apexes of a triangle and a light emitter and two cameras disposed apart from the pointer and detects the three-dimensional position and the rotation angle about an orthogonal coordinate axis of the pointer based on the two images formed by receiving the light of the light emitter, which is reflected by the three reflectors, by the two cameras: and
the control means sets the three-dimensional position and the rotation angle about an orthogonal coordinate is of the cross section by determining the three-dimensional position and the rotation angle about the orthogonal coordinate axis of the portion subjected to measurement on based on the three-dimensional position and the rotation angle about an orthogonal coordinate axis of the pointer detected by the position detection means.

8. A magnetic resonance imaging apparatus according to claim 6 or 7, wherein the operation means has correlation data created by previously measuring the correlation between the movement of the portion subjected to measurement moved by body movement and the movement of the pointer and determines the three-dimensional position and/or the rotation angle about orthogonal coordinate axes of the portion subjected to measurement from the three-dimensional position and/or the rotation angle about an orthogonal coordinate axis detected by the position detection means based on the correlation data.

9. A magnetic resonance imaging apparatus according to claim 1, wherein the operation means determines first magnetic resonance signals as a reference and second magnetic resonance signals after body movement and determines a temperature change distribution by calculating the difference between the temperature distributions of the determined sets of the portion subjected to measurements.

10. A magnetic resonance imaging apparatus according to claim 9, wherein the body movement detection means includes position detection means that comprises the pointer having the three reflectors for reflecting light disposed discretely in contact with a body surface near to the portion subjected to measurement and a light emitter and two cameras disposed apart from the pointer and detects the three-dimensional position and the rotation angle about an orthogonal coordinate axis of the pointer based on the two images formed by receiving the light of the light emitter, which is reflected by the three reflectors, by the two cameras; and
the control means sets the three-dimensional position and the rotation angle about an orthogonal coordinate axis of the cross section by determining the three-dimensional position and the rotation angle about an orthogonal coordinate axis of the portion subjected to measurement based on the three-dimensional position and the rotation angle about an orthogonal coordinate axis of the pointer detected by the position detection means.

11. A magnetic resonance imaging apparatus according to claim 10, wherein the operation means has correlation data created by previously measuring the correlation between the movement of the portion subjected to measurement moved by body movement and the movement of the pointer and determines the three-dimensional position and the rotation angle about an orthogonal coordinate axis of the portion subjected to measurement from the three-dimensional position and the rotation angle about an orthogonal coordinate axis detected by the position detection means based on the correlation data.

12. A magnetic resonance imaging apparatus according to claim 1 or 2, wherein the portion subjected to measurement is a portion including the extreme end of a needle device inserted in the examinee; and
the operation means has a function for determining the temperature distribution of the portion subjected to measurement using the respective sets of the nuclear magnetic resonance signals and determining a temperature change distribution by calculating the difference between the temperature distributions of the thus determined respective sets of the portion subjected to measurement.

13. A magnetic resonance imaging apparatus according to claim 13, wherein the body movement detection means comprises position detection means that comprises a pointer having reflectors discretely attached to the portions of the needle device disposed outside of the body and a light emitter and two cameras disposed apart from the pointer and detects the three-dimensional position of the pointer based on the two images formed by receiving the light of the light emitter, which is reflected by the three reflectors, by the two cameras; and
the control means determines the three-dimensional positions of the extreme end of the needle device based on the three-dimensional position of the pointer detected by the position detection means and sets the three-dimensional position of the cross section such that the cross section includes the axis of the needle device as well as the three-dimensional position of the cross section agree with that of the needle device.

14. A magnetic resonance imaging apparatus according to any of claims 9 to 13, wherein the operation means includes a function for forming the temperature change distribution of the portion subjected to measurement as an image and displaying the image on a display screen.

15. A magnetic resonance imaging apparatus according to claim 1 or 2, wherein the operation means has an image processing function for rearranging tomograms including the portion subjected to measurement using the respective sets of the nuclear magnetic resonance signals and determining the differential image of the respective sets of the rearranged tomograms.

16. A magnetic resonance imaging apparatus according to claim 1 or 2, wherein the operation means has an image processing function for rearranging blood vessel images including the portion subjected to measurement using the respective sets of the nuclear magnetic resonance signals and determining the differential image of the respective sets of the rearranged blood vessel images.

17. A magnetic resonance imaging apparatus according to claim 15 or 16, wherein the body movement detection means includes position detection means that comprises a pointer having the three reflectors for reflecting light disposed discretely in contact with a body surface near to the portion subjected to measurement and a light emitter and two cameras disposed apart from the pointer and detects the three-dimensional position and the rotation angle about an orthogonal coordinate axis of the pointer based on the two Images formed by receiving the light of the light emitter, which is reflected by the three reflectors, by the two cameras; and
the control means sets the three-dimensional position and the rotation angle about an orthogonal coordinate axis of the cross section by determining the three-dimensional position and the rotation angle about an orthogonal coordinate axis of the portion subjected to measurement based on the three-dimensional position and the rotation angle about an orthogonal coordinate axis of the pointer detected by the position detection means.

18. A magnetic resonance imaging apparatus according to claim 17, wherein the operation means has correlation data created by previously detecting the correlation between the movement of the portion subjected to measurement moved by body movement and the movement of the pointer and determines the three-dimensional position and the rotation angle about an orthogonal coordinate axis of the portion subjected to measurement from the three-dimensional position and the rotation angle about an orthogonal coordinate axis detected by the position detection means based on the correlation data.

19. A magnetic resonance imaging method which comprises the steps of obtaining time series images of a measuring cross section including a portion subjected to measurement of an examinee using magnetic resonance imaging, and obtaining diagnosis information by comparing magnetic resonance signals related to the plurality of the measuring cross sections obtained in an operation process, wherein the body movement of the examinee is detected and a position of the cross section is set so as to include the portion subjected to measurement in conformity with the detected body movement.

20. A magnetic resonance imaging method according to claim 19, wherein the movement of the body surface or the movement of the portion moved in relation to the body surface is detected by detecting the three-dimensional position and the rotation angle about an orthogonal coordinate axis of the body surface or the portion.
